(19) United States
(12) Patent Application Publication
(10) Pub. No.: US 2024/0068006 A1
(43) Pub. Date: Feb. 29, 2024

(54) METHOD FOR ASSESSING DRUG-RESISTANT KLEBSIELLA PNEUMONIAE AND DRUG-RESISTANT KLEBSIELLA PNEUMONIAE ASSESSING SYSTEM

(71) Applicant: China Medical University, Taichung City (TW)

(72) Inventors: Der-Yang Cho, Taichung City (TW); Po-Ren Hsueh, Taichung City (TW); Jiaxin Yu, Taichung City (TW); Ni Tien, Taichung City (TW); Hsiu Hsien Lin, Taichung City (TW); Chia-Fong Cho, Taichung City (TW)

(73) Assignee: China Medical University, Taichung City (TW)

(21) Appl. No.: 18/154,906

(22) Filed: Jan. 16, 2023

(30) Foreign Application Priority Data
Aug. 29, 2022 (TW) 111132547

(51) Int. Cl. C12Q 1/10; G01N 33/68

(52) U.S. Cl. CPC C12Q 1/10; G01N 33/6848; G01N 2333/986

(57) ABSTRACT

A method for assessing drug-resistant Klebsiella pneumoniae includes the following steps. A test sample is provided, wherein the test sample includes a Klebsiella pneumoniae. A spectrum analysis step is performed, wherein the test sample is detected by a mass spectrometry method so as to obtain a target mass spectrum data. An assessing step for drug-resistant Klebsiella pneumoniae is performed, wherein the target mass spectrum data is analyzed so as to assess whether the Klebsiella pneumoniae is resistant to a carbapenem antibiotic or a colistin or not. When the Klebsiella pneumoniae is resistant to the carbapenem antibiotic, the target mass spectrum data includes a first anti-carbapenem feature mark, and when the Klebsiella pneumoniae is resistant to the colistin, the target mass spectrum data includes a first anti-colistin feature mark.

### RELATED APPLICATIONS

This application claims priority to Taiwan Application Serial Number 111132547, filed Aug. 29, 2022, which is herein incorporated by reference.

### BACKGROUND

### Technical Field

The present disclosure relates to a medical information analysis method and a system thereof. More particularly, the present disclosure relates to a method for assessing drug-resistant Klebsiella pneumoniae and a drug-resistant Klebsiella pneumoniae assessing system.

### Description of Related Art

The carbapenem antibiotic is a general name of a class of broad-acting antibiotics and is often used as a last-line antibiotic to treat serious bacterial infections. However, the infection caused by carbapenem-no susceptible Enterobacteriaceae (CnSE) has appeared in the current clinical and has a significant impact on the public health.

In the different resistance mechanisms of carbapenem antibiotics, the ability to synthesize the carbapenemase is critical. Because the gene encoding the carbapenemase is located in the plasmid, and the plasm id can be spread between bacteria by conjugation, the prevalence of carbapenemase-producing Enterobacteriaceae (CPE) is increasing day by day.

In order to inhibit the prevalence of CPE, a variety of carbapenemase inhibitors are used in clinical treatments. However, different kinds of carbapenemases are found in clinical, and when the bacterium produce different carbapenemases, the single carbapenemase inhibitor can have other inhibitory effects on different kinds of carbapenemases. In detail, Klebsiella pneumoniae carbapenemase (KPC) encoded by the bla_{KPC} gene is classified as Ambler class A carbapemease. Metallo-beta-lactamase (MBL) including imipenemase MBL (IMP) encoded by the bla_{IMP} gene, Verona integron-encoded MBL (VIM) encoded by the bla_{VIM} gene or New Delhi MBL (NDM) encoded by the bla_{NDM} gene are classified as Ambler class B carbapemease. Oxacillinase-type carbapenemase (which can hydrolyze oxacillin; OXA) encoded by the bla_{OXA} gene is classified as Ambler class D carbapemease. Furthermore, the KPC, NDM, IMP, VIM and certain OXA-like enzymes are the most commonly identified variant carbapenemases that have spread world-wide among Enterobacterales. The OXA-48-like carbapenemase is encoded by the bla_{oxA-48}, the bla_{oxA-162}, the bla_{oxA-181}, the bla_{OXA-204}, the bla_{oxA-232} or the bla_{OXA-244} genes. The OXA-48-like carbapenemase is distinguished by the substitution of one to five specific amino acids in the β5-β6 loop of the enzyme, leading to different ability to hydrolyze carbapenems.

Further, the colistin, which is regarded as the last choice among the currently used antibiotics, can be used to treat the infections of antibiotic-resistant Gram-negative bacterium, such as carbapenem-resistant Enterobacteriaceae (CRE) and carbapenem-resistant Acinetobacter baumannii (CRAB). However, the colistin has nephrotoxicity and neurotoxicity, the elderly and those with renal insufficiency should be used with caution, resulting in the clinical use of the colistin being very careful and strict.

In the current clinical, the gold standard to test the drug susceptibility of the colistin is the broth dilution method (BMD). However, the testing steps of the broth dilution method are very complicated, and the requirements thereof are also very strict. Accordingly, the broth dilution method is difficult to be applied as a routine inspection method in medical institutions.

Therefore, how to provide a rapid and accurate method to identify whether a microorganism is sensitive to the carbapenem antibiotics or the colistin or not, so as to rapidly and accurately provide an appropriate treatment strategy, has become the goal of the relevant academic and industry development.

### SUMMARY

According to one aspect of the present disclosure, a method for assessing drug-resistant Klebsiella pneumoniae includes the following steps. A test sample is provided, wherein the test sample includes a Klebsiella pneumoniae. A spectrum analysis step is performed, wherein the test sample is detected by a mass spectrometry method so as to obtain a target mass spectrum data, and the target mass spectrum data is a mass spectrum data of the Klebsiella pneumoniae. An assessing step for drug-resistant Klebsiella pneumoniae is performed, wherein the target mass spectrum data is analyzed so as to assess whether the Klebsiella pneumoniae is resistant to a carbapenem antibiotic or not. When the Klebsiella pneumoniae is resistant to the carbapenem antibiotic, the target mass spectrum data includes a first anti-carbapenem feature mark, and a mass-to-charge ratio of the first anti-carbapenem feature mark ranges from 4,520 to 4,529 daltons.

According to another aspect of the present disclosure, a method for assessing drug-resistant Klebsiella pneumoniae includes the following steps. A test sample is provided, wherein the test sample includes a Klebsiella pneumoniae. A spectrum analysis step is performed, wherein the test sample is detected by a mass spectrometry method so as to obtain a target mass spectrum data, and the target mass spectrum data is a mass spectrum data of the Klebsiella pneumoniae. An assessing step for drug-resistant Klebsiella pneumoniae is performed, wherein the target mass spectrum data is analyzed so as to assess whether the Klebsiella pneumoniae is resistant to a colistin or not. When the Klebsiella pneumoniae is resistant to the colistin, the target mass spectrum data includes a first anti-colistin feature mark, and a mass-to-charge ratio of the first anti-colistin feature mark ranges from 4,170 to 4,179 daltons.

According to further another aspect of the present disclosure, a drug-resistant Klebsiella pneumoniae assessing system includes a non-transitory machine readable medium and a processor. The non-transitory machine readable medium is for storing a target mass spectrum data, wherein the target mass spectrum data is obtained by detecting a test sample with a mass spectrometry method, and the test sample includes a Klebsiella pneumoniae. The processor is signally connected to the non-transitory machine readable medium, and the processor includes an analyzing module. The analyzing module is for analyzing the target mass spectrum data by a drug susceptibility analysis program so as to output a feature set, wherein the drug susceptibility analysis program is for assessing whether the Klebsiella pneumoniae is resistant to a carbapenem antibiotic or a colistin or not based on the feature set. The feature set includes a first anti-carbapenem feature mark and a first anti-colistin feature mark, a mass-to-charge ratio of the first anti-carbapenem feature mark ranges from 4,520 to 4,529 daltons, and a mass-to-charge ratio of the first anti-colistin feature mark ranges from 4,170 to 4,179 daltons. When the Klebsiella pneumoniae is resistant to the carbapenem antibiotic, the target mass spectrum data includes the first anti-carbapenem feature mark. When the Klebsiella pneumoniae is resistant to the colistin, the target mass spectrum data includes the first anti-colistin feature mark.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1 is a flow chart of a method for assessing drug-resistant Klebsiella pneumoniae according to one embodiment of the present disclosure.

FIG. 2 is a block diagram of a drug-resistant Klebsiella pneumoniae assessing system according to another embodiment of the present disclosure.

FIG. 3 is a flow chart of a method for assessing drug-resistant Klebsiella pneumoniae of the 1st Example.

FIG. 4 is a block diagram of a drug-resistant Klebsiella pneumoniae assessing system of the 2nd Example.

FIG. 5 shows the analysis results of the contributions of different feature marks to the drug susceptibility analysis program when assessing the susceptibility of the Klebsiella pneumoniae.

FIG. 6 shows the mass spectrometry data of different types of Klebsiella pneumoniae.

FIG. 7 is a pseudogel plot of all samples used to assess the susceptibility of the Klebsiella pneumoniae to the carbapenem antibiotics.

FIG. 8 shows the mass spectrometry data of different types of CnSKP.

FIG. 9 is a pseudogel plot of all samples used to assess the CnSKP of different Ambler classifications.

FIG. 10 shows the analysis results of the contributions of different feature marks to the drug susceptibility analysis program when assessing the susceptibility of the Klebsiella pneumoniae.

FIG. 11 shows the mass spectrometry data of different types of colistin-resistant Klebsiella pneumoniae.

FIG. 12 is a pseudogel plot of all samples used to assess the susceptibility of Klebsiella pneumoniae to the colistin.

### DETAILED DESCRIPTION

The various embodiments of the invention are discussed in more detail below. However, the embodiments may be specific to various applications of the inventive concept and may be practiced in a variety of specific contexts. The particular embodiments are for illustrative purposes only and are not limited to the scope of the disclosure.

[Method for Assessing Drug-Resistant Klebsiella pneumoniae of the Present Disclosure]

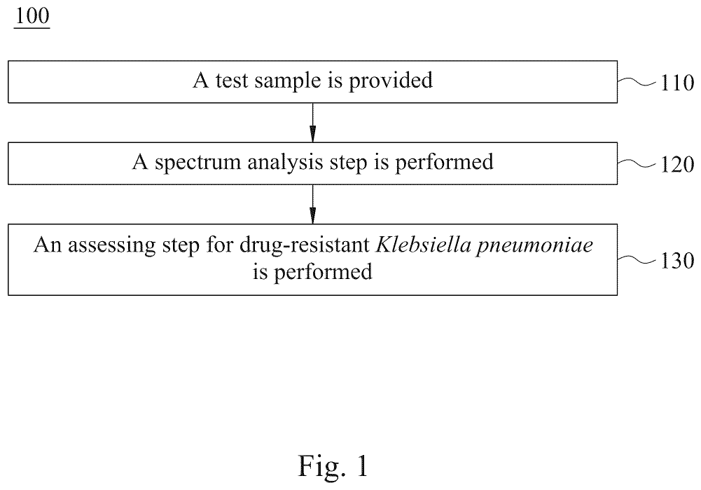

Reference is made to FIG. 1, which is a flow chart of a method 100 for assessing drug-resistant Klebsiella pneumoniae according to one embodiment of the present disclosure. The method 100 for assessing drug-resistant Klebsiella pneumoniae includes Step 110, Step 120 and Step 130.

In Step 110, a test sample is provided. The test sample includes a Klebsiella pneumoniae. Klebsiella pneumoniae is a bacteria belonging to the genus Klebsiella of Enterobacteriaceae, and Klebsiella pneumoniae can cause pneumonia, urinary tract infection, bacteremia and other infectious diseases in the humans. According to the current research, the prevalence of the Klebsiella pneumoniae which is insensitive to the carbapenem antibiotics is increasing year by year, so that the Klebsiella pneumoniae is used as the target microorganism in the method 100 for assessing drug-resistant Klebsiella pneumoniae of the present disclosure so as to respond to the increasing number of the related clinical cases.

In Step 120, a spectrum analysis step is performed, wherein the test sample is detected by a mass spectrometry method so as to obtain a target mass spectrum data, and the target mass spectrum data is a mass spectrum data of the Klebsiella pneumoniae. In detail, the mass spectrometry method used in the present disclosure is Matrix Assisted Laser Desorption Ionization Time-of-Flight mass spectrometry (“MALDI-TOF” hereafter). In greater detail, MALDI-TOF can mix the sample of different types (liquid or solid) with the detection reagent (substrate), and then the laser is used to excite the sample to form gas-phase ions. Then, the mass-to-charge ratio of each of the gas-phase ions is detected by a mass spectrometer and then converted into a mass spectrum data. In the clinical application, the mass spectrum data of the sample can be compared with the mass spectrometry database of the microorganisms established by the same mass spectrometry method, so the strains of the microorganisms can be identified based on the principle that the mass spectrum data of the same microorganism is identical. Accordingly, by using the MALDI-TOF method to detect the test sample to obtain the mass spectrum data of the Klebsiella pneumoniae, the method 100 for assessing drug-resistant Klebsiella pneumoniae of the present disclosure can be close to the current clinical process used to identify microorganisms, and it has not only a high market acceptance but also a high assessment accuracy in the related application.

In Step 130, an assessing step for drug-resistant Klebsiella pneumoniae is performed, wherein the target mass spectrum data is analyzed so as to assess whether the Klebsiella pneumoniae is resistant to a carbapenem antibiotic or a colistin or not.

In detail, when the Klebsiella pneumoniae is resistant to the carbapenem antibiotic, the target mass spectrum data includes a first anti-carbapenem feature mark, and a mass-to-charge ratio of the first anti-carbapenem feature mark ranges from 4,520 to 4,529 daltons. In other words, when the carbapenem antibiotic has no inhibitory effect on the Klebsiella pneumoniae in the test sample, the target mass spectrum data of the Klebsiella pneumoniae of the test sample has a peak at the mass-to-charge ratio of 4,520 to 4,529 daltons.

Further, when the Klebsiella pneumoniae is resistant to the colistin, the target mass spectrum data includes a first anti-colistin feature mark, and a mass-to-charge ratio of the first anti-colistin feature mark ranges from 4,170 to 4,179 daltons. In other words, when the colistin has no inhibitory effect on the Klebsiella pneumoniae in the test sample, the target mass spectrum data of the Klebsiella pneumoniae of the test sample has a peak at the mass-to-charge ratio of 4,170 to 4,179 daltons.

In greatly detail, when the target mass spectrum data of the Klebsiella pneumoniae is analyzed, if the target mass spectrum data includes the first anti-carbapenem feature mark or the first anti-colistin feature mark, the Klebsiella pneumoniae in the test sample can be assessed to be resistant to the carbapenem antibiotic or the colistin. Then, a drug susceptibility assessing result of the Klebsiella pneumoniae will be output accordingly so as to facilitate the formulation of the medical strategy in clinical. Therefore, the method 100 for assessing drug-resistant Klebsiella pneumoniae of the present disclosure can be effectively used to assess whether the Klebsiella pneumoniae is resistant to the carbapenem antibiotic or the colistin or not based on the mass spectrometry data thereof, and the method 100 for assessing drug-resistant Klebsiella pneumoniae has the potential application in relevant markets.

Furthermore, according to the β-lactamase classification proposed by Ambler et al. (“Ambler classification” hereafter), the Klebsiella pneumoniae with the genes encoding the carbapenemases can be classified into Ambler class A, Ambler class B and Ambler class D. In detail, the Klebsiella pneumoniae including a bla_{KPC} gene (encoding Klebsiella pneumoniae carbapenemase; KPC) is classified as Ambler class A, the Klebsiella pneumoniae including a bla_{NDM} gene (encoding New Delhi metallo-β-lactamase; NDM), a bla_{VIM} gene (encoding Verona integron-encoded metallo-β lactamase; VIM) or a bla_{IMP} gene (which is active on imipenem; IMP) is classified as Ambler class B, and the Klebsiella pneumoniae including a bla_{OXA} gene (which can hydrolyze oxacillin; OXA) is classified as Ambler class D. Accordingly, when the target mass spectrum data of the present disclosure includes the first anti-carbapenem feature mark, based on the result of genotype comparison, the Klebsiella pneumoniae including the bla_{KPC} gene and a Klebsiella pneumoniae carbapenemase is classified as the Klebsiella pneumoniae of Ambler class A, and the Klebsiella pneumoniae is a carbapenem resistant Klebsiella pneumoniae.

Further, when the Klebsiella pneumoniae is resistant to the carbapenem antibiotic, the target mass spectrum data can further include a second anti-carbapenem feature mark, and a mass-to-charge ratio of the second anti-carbapenem feature mark can range from 6,150 to 6,159 daltons. Furthermore, when the target mass spectrum data includes the second anti-carbapenem feature mark, based on the result of genotype comparison, the Klebsiella pneumoniae includes the bla_{NDM} gene, the bla_{VIM} gene or the bla_{IMP} gene and includes a metallo-β-lactamases (MBL). Particularly, the metallo-β-lactamases can be imipenemase MBL (IMP), New Delhi MBL (NDM), or Verona integron-encoded MBL (VIM), and the Klebsiella pneumoniae is classified as the Klebsiella pneumoniae of Ambler class B.

Further, when the Klebsiella pneumoniae is resistant to the carbapenem antibiotic, the target mass spectrum data can further include a third anti-carbapenem feature mark, and a mass-to-charge ratio of the third anti-carbapenem feature mark can range from 2,180 to 2,189 daltons. Furthermore, when the target mass spectrum data includes the third anti-carbapenem feature mark, based on the result of genotype comparison, the Klebsiella pneumoniae including the blaₒₓₚ, gene and an oxacillinase-type carbapenemase is classified as the Klebsiella pneumoniae of Ambler class D. Specifically, the oxacillinase-type carbapenemase can be OXA-48-like carbapenemases, which is encoded by bla_{OXA-48-like} gene.

Moreover, when the Klebsiella pneumoniae is resistant to the colistin, the target mass spectrum data can further include a second anti-colistin feature mark, and a mass-to-charge ratio of the second anti-colistin feature mark can range from 7,320 to 7,329 daltons.

[Drug-Resistant Klebsiella pneumoniae Assessing System of the Present Disclosure]

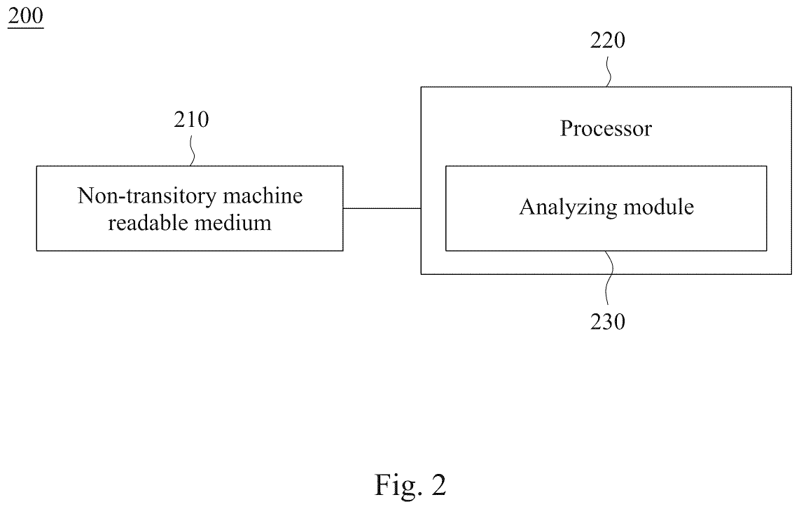

Reference is made to FIG. 2, which is a block diagram of a drug-resistant Klebsiella pneumoniae assessing system 200 according to another embodiment of the present disclosure. The drug-resistant Klebsiella pneumoniae assessing system 200 includes a non-transitory machine readable medium 210 and a processor 220.

The non-transitory machine readable medium 210 is for storing a target mass spectrum data, wherein the target mass spectrum data is obtained by detecting a test sample with a mass spectrometry method, and the test sample includes a Klebsiella pneumoniae.

The processor 220 is signally connected to the non-transitory machine readable medium 210, and the processor 220 includes an analyzing module 230. The analyzing module 230 is for analyzing the target mass spectrum data by a drug susceptibility analysis program so as to output a feature set, and the drug susceptibility analysis program is for assessing whether the Klebsiella pneumoniae is resistant to a carbapenem antibiotic or a colistin or not based on the feature set.

In detail, the feature set includes a first anti-carbapenem feature mark and a first anti-colistin feature mark. A mass-to-charge ratio of the first anti-carbapenem feature mark ranges from 4,520 to 4,529 daltons, and a mass-to-charge ratio of the first anti-colistin feature mark ranges from 4,170 to 4,179 daltons. When the Klebsiella pneumoniae is resistant to the carbapenem antibiotic, the target mass spectrum data includes the first anti-carbapenem feature mark. When the Klebsiella pneumoniae is resistant to the colistin, the target mass spectrum data includes the first anti-colistin feature mark. Further, when the target mass spectrum data includes the first anti-carbapenem feature mark, based on the result of genotype comparison, the Klebsiella pneumoniae can include a Klebsiella pneumoniae carbapenemase, which is encoded by a bla_{kpc} gene.

Further, the feature set can further include a second anti-carbapenem feature mark. When the Klebsiella pneumoniae is resistant to the carbapenem antibiotic, the target mass spectrum data can further include the second anti-carbapenem feature mark, and a mass-to-charge ratio of the second anti-carbapenem feature mark can range from 6,150 to 6,159 daltons. Furthermore, when the target mass spectrum data includes the second anti-carbapenem feature mark, based on the result of genotype comparison, the Klebsiella pneumoniae can include a bla_{NDM} gene, a bla_{VIM} gene or a bla_{IMP} gene. In other words, when the target mass spectrum data includes the second anti-carbapenem feature mark, the Klebsiella pneumoniae includes the metallo-β-lactamases, and the metallo-β-lactamases can be imipenemase MBL, New Delhi MBL, or Verona integron-encoded MBL, which are encoded by bla_{NDM} gene, bla_{VIM} gene or bla_{IMP} gene, respectively.

Further, the feature set can further include a third anti-carbapenem feature mark. When the Klebsiella pneumoniae is resistant to the carbapenem antibiotic, the target mass spectrum data can further include the third anti-carbapenem feature mark, and a mass-to-charge ratio of the third anti-carbapenem feature mark can range from 2,180 to 2,189 daltons. Furthermore, when the target mass spectrum data includes the third anti-carbapenem feature mark, based on the result of genotype comparison, the Klebsiella pneumoniae can include a bla_{oxA} gene encoding an oxacillinase-type carbapenemase. Specifically, the oxacillinase-type carbapenemase can be OXA-48-like carbapenemases, which is encoded by bla_{oxA-48-like} gene.

Further, the feature set can further include a second anti-colistin feature mark. When the Klebsiella pneumoniae is resistant to the colistin, the target mass spectrum data can further include the second anti-colistin feature mark, and a mass-to-charge ratio of the second anti-colistin feature mark can range from 7,320 to 7,329 daltons.

The method for assessing drug-resistant Klebsiella pneumoniae and the drug-resistant Klebsiella pneumoniae assessing system of the present disclosure will be further exemplified by performing the following specific embodiments. However, the present disclosure should not be limited to these practical details thereof, that is, in some embodiments, these practical details are used to describe how to implement the materials and methods of the present disclosure and are not necessary.

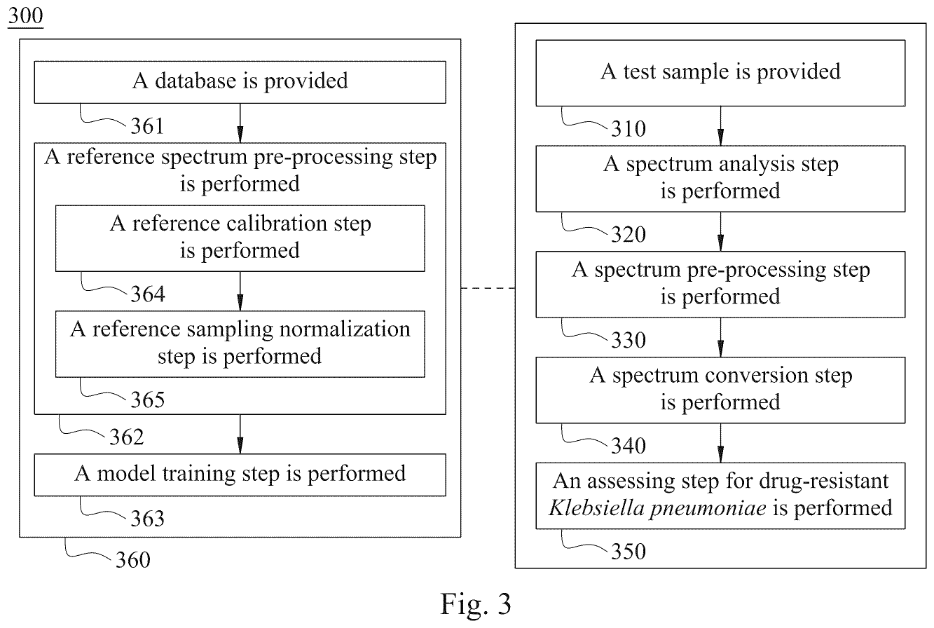

Reference is made to FIG. 3, which is a flow chart of a method 300 for assessing drug-resistant Klebsiella pneumoniae of the 1st Example. The method 300 for assessing drug-resistant Klebsiella pneumoniae includes Step 310, Step 320, Step 330, Step 340 and Step 350, wherein Step 310 and Step 320 are the same to Step 110 and Step 120 of FIG. 1, so that the details thereof will not be described herein again.

In Step 330, a spectrum pre-processing step is performed, wherein the target mass spectrum data is pre-processed so as to obtain a processed target mass spectrum data, and the spectrum pre-processing step can include a calibration step and a sampling normalization step. In the calibration step, the signals of the target mass spectrum data will be smoothed so as to remove the background noise thereof. In the sampling normalization step, a temporal resolution value of the target mass spectrum data is adjusted so as to obtain the processed target mass spectrum data.

In greatly detail, in the sampling normalization step, the raw signal resolution and the sampling frequency of the target mass spectrum data processed by the calibration step will be checked whether there is an inconsistency or not. If so, the target mass spectrum data will be resampled in the sampling normalization step so as to make the temporal resolution values thereof consistent. Then, a baseline correction will be performed by the top-hat method, and the following Formula (I) is used to normalize the signal intensity so as to make the signal resolution consistent. Formula (I) is shown below:

z=(x−μ)/σ Formula (I);

wherein “z” represents z-score, “x” represents the mass-to-charge ratio intensity of each point of the target mass spectrum data, “μ” represents the average signal intensity of the target mass spectrum data, and “σ” represents the standard deviation of intensity of the target mass spectrum data. After normalizing the intensity of the target mass spectrum data, the mass-to-charge ratio intensity data with negative z-scores represents that the subtle signals or the noise are existed, and the said target mass spectrum data will be further removed. Thus, the processed target mass spectrum data without the background noise and the sampling frequency and the intensity thereof being normalized can be obtained.

In Step 340, a spectrum conversion step is performed, wherein the processed target mass spectrum data is processed by a mass-to-charge ratio conversing method so as to obtain a normalized target mass spectrum data. Further, in the spectrum conversion step, a data interval value of the normalized target mass spectrum data can be further adjusted by a binning method so as to prevent the peaks of the normalized target mass spectrum data from shifting due to the effects of the isotopes in the mass spectrometry method, but the present disclosure is not limited thereto.

In Step 350, an assessing step for drug-resistant Klebsiella pneumoniae is performed, wherein the normalized target mass spectrum data is analyzed so as to assess whether the Klebsiella pneumoniae is resistant to a carbapenem antibiotic or a colistin or not.

As shown in FIG. 3, in the 1st Example, the method 300 for assessing drug-resistant Klebsiella pneumoniae can further include Step 360. In Step 360, a model establishing step is performed, wherein the model establishing step is for establishing an antibiotic resistance assessing classifier, and the antibiotic resistance assessing classifier is for analyzing the importance of different characteristic feature marks for drug susceptibility assessment. The model establishing step includes Step 361, Step 362 and Step 363.

In Step 361, a database is provided, wherein the database includes a plurality of reference mass spectrum data, and each of the reference mass spectrum data is a mass spectrum data of a Klebsiella pneumoniae. Further, the reference mass spectrum data can be MALDI-TOF mass spectrum data so as to be close to the current clinical process used to identify microorganisms.

In Step 362, a reference spectrum pre-processing step is performed, wherein all of the reference mass spectrum data are pre-processed so as to obtain a plurality of normalized reference mass spectrum data. In detail, the reference spectrum pre-processing step can include Step 364 and Step 365.

In Step 364, a reference calibration step is performed, wherein a background noise of each of the reference mass spectrum data is removed. In detail, before reference calibration step is performed, each of the reference mass spectrum data will be examined initially so as to exclude the mass spectrum data including blank portions or error formats. Then, each of the reference mass spectrum data will be smoothed so as to remove the background noise thereof.

In Step 365, a reference sampling normalization step is performed, wherein a temporal resolution value of each of the reference mass spectrum data is adjusted so as to obtain the plurality of normalized reference mass spectrum data. In detail, in the reference sampling normalization step, the raw signal resolution and the sampling frequency of all of the reference mass spectrum data will be respectively checked so as to check whether there is an inconsistency or not. If so, all the reference mass spectrum data will be resampled so as to make the temporal resolution values thereof consistent. Further, the baseline of each of the reference mass spectrum data will be corrected by the top-hat method and then normalized according to the aforementioned Formula (I), so that the signal resolution of all the reference mass spectrum data can be consistent. The details of Formula (I) are shown in the aforementioned paragraph and will not be described herein again.

In Step 363, a model training step is performed, wherein the plurality of the normalized reference mass spectrum data are trained to achieve a convergence by an algorithm classifier so as to obtain the antibiotic resistance assessing classifier. Preferably, the algorithm classifier can be LightGBM (Light Gradient Boosting Machine) algorithm classifier, CatBoost algorithm classifier, XGBoost (Extreme Gradient Boosting) algorithm classifier, Gradient Boosting algorithm classifier or other algorithm classifiers based on the decision tree algorithm, but the present disclosure is not limited thereto.

In detail, the reference mass spectrum data collected clinically can be analyzed by the antibiotic resistance assessing classifier so as to find the characteristic feature marks which can be used as the feature marks of the Klebsiella pneumoniae being resistant to the carbapenem antibiotic or the feature marks of the Klebsiella pneumoniae being resistant to the colistin, and the importance of different feature marks can be further assessed. Thus, at least one of the feature marks obtained therefrom will be used in the method for assessing drug-resistant Klebsiella pneumoniae and the drug-resistant Klebsiella pneumoniae assessing system of the present disclosure as the base to assess whether the Klebsiella pneumoniae is resistant to the carbapenem antibiotic or the colistin or not.

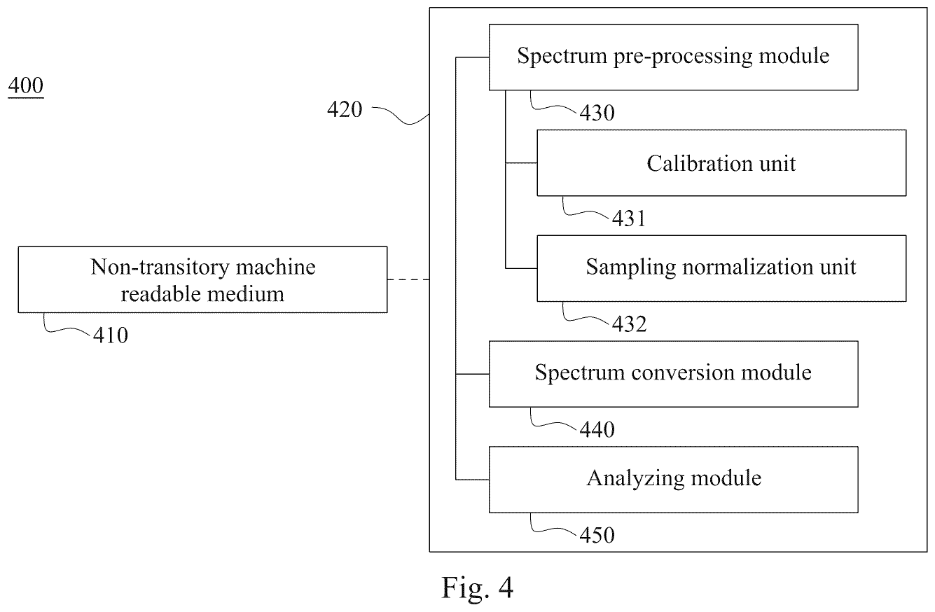

Reference is made to FIG. 4, which is a block diagram of a drug-resistant Klebsiella pneumoniae assessing system 400 of the 2nd Example. The drug-resistant Klebsiella pneumoniae assessing system 400 includes a non-transitory machine readable medium 410 and a processor 420.

The non-transitory machine readable medium 410 is for storing a target mass spectrum data, wherein the target mass spectrum data is obtained by detecting a test sample with a mass spectrometry method, and the test sample includes a Klebsiella pneumoniae.

The processor 420 is signally connected to the non-transitory machine readable medium 410, and the processor 420 includes a spectrum pre-processing module 430, a spectrum conversion module 440 and an analyzing module 450.

The spectrum pre-processing module 430 is for pre-processing the target mass spectrum data so as to obtain a processed target mass spectrum data. In detail, the spectrum pre-processing module 430 can include a calibration unit 431 and a sampling normalization unit 432. The calibration unit 431 is for removing a background noise of the target mass spectrum data, and the sampling normalization unit 432 is for adjusting a temporal resolution value of the target mass spectrum data, so that the processed target mass spectrum data can be obtained.

The spectrum conversion module 440 is for processing the processed target mass spectrum data by a mass-to-charge ratio conversing method so as to obtain a normalized target mass spectrum data.

The analyzing module 450 is for analyzing the normalized target mass spectrum data by a drug susceptibility analysis program so as to output a feature set, and the drug susceptibility analysis program is for assessing whether the Klebsiella pneumoniae is resistant to a carbapenem antibiotic or a colistin or not based on the feature set. In detail, the feature set includes a first anti-carbapenem feature mark and a first anti-colistin feature mark, wherein a mass-to-charge ratio of the first anti-carbapenem feature mark ranges from 4,520 to 4,529 daltons, and a mass-to-charge ratio of the first anti-colistin feature mark ranges from 4,170 to 4,179 daltons. When the Klebsiella pneumoniae is resistant to the carbapenem antibiotic, the target mass spectrum data includes the first anti-carbapenem feature mark. When the Klebsiella pneumoniae is resistant to the colistin, the target mass spectrum data includes the first anti-colistin feature mark.

Further, the processing details of the spectrum conversion module 440 and the analyzing module 450 are similar to that of Step 340 and Step 350, so that the details thereof will not be described again. Furthermore, the establishing details of the antibiotic resistance assessing classifier are described in Step 361, Step 362, Step 363, Step 364 and Step 365 of Step 360, so that the details thereof will also not be described again.

Therefore, the method 300 for assessing drug-resistant Klebsiella pneumoniae and the drug-resistant Klebsiella pneumoniae assessing system 400 of the present disclosure can effectively assess whether the Klebsiella pneumoniae is resistant to the carbapenem antibiotic or the colistin or not based on that whether the mass spectrum data of the Klebsiella pneumoniae includes the first anti-carbapenem feature mark or the first anti-colistin feature mark. Accordingly, the time required for the conventional microbial culture, the identification and the antibiotic susceptibility testing can be effectively reduced, and it is favorable for formulating the medical strategy for the drug-resistant Klebsiella pneumoniae, and the present disclosure has the potential application in clinical.

### Testing Example

In order to assess the efficacy of each of the first anti-carbapenem feature mark, the second anti-carbapenem feature mark, the third anti-carbapenem feature mark, the first anti-colistin feature mark and the second anti-colistin feature mark of the present disclosure for assessing whether the Klebsiella pneumoniae is resistant to the carbapenem antibiotic or the colistin or not, the following specific testing examples will be further exemplified.

I. Database

The database of the present test includes 3,938 of the reference mass spectrum data, wherein the reference mass spectrum data are obtained after the samples infected by the Klebsiella pneumoniae are processed by a conventional sample processing method in the clinical laboratory. The Klebsiella pneumoniae used in the present test includes carbapenem-susceptible Klebsiella pneumoniae (“CSKP” hereafter), carbapenem-nonsusceptible Klebsiella pneumoniae (“CnSKP” hereafter), colistin-resistant Klebsiella pneumoniae (“CoIRKP” hereafter) and colistin-intermediate Klebsiella pneumoniae (“CoIIKP” hereafter). Generally, the susceptibilities of carbapenem antibiotic of the Klebsiella pneumoniae is determined using the broth microdilution method and is interpreted according to the Clinical and Laboratory Standard Institute (CLSI) guidelines to determine the minimum inhibitory concentrations (MICs) of antibiotics against the Klebsiella pneumoniae isolates. Carbapenem antibiotics with a carbapenem core structure, such as imipenem, meropenem, doripenem, and ertapenem, have broad-spectrum in vitro activity. When a microorganism is not sensitive to any of the carbapenems, it is judged to have carbapenem resistance. In detail, in the present disclosure, the MALDI-TOF data with the label of carbapenem susceptibility is according to the results from Phoenix susceptibility system (Becton-Dickinson Diagnostic Systems, Sparks, MD, USA).

Further, the ColIKP is identified according to the specifications in the guidelines of CLSI published in 2020. When a breakpoint of the MIC of the colistin is less than or equal to 2 mg/L, the ColIKP belongs to the intermediate type (I), and when a breakpoint of the minimum inhibitory concentration of the colistin is larger than or equal to 4 mg/L, the ColIKP belongs to the resistant type (R). The numbers of the reference mass spectrum data of CSKP, CnSKP, CoIRKP and ColIKP are shown in Table 1.

TABLE 1

| Klebsiella pneumoniae | Sample number |
|---|---|
| CSKP | 1,462 |
| CnSKP | 830 |
| ColRKP | 54 |
| ColIKP | 1,592 |

Further, in the present test, the Klebsiella pneumoniae corresponding to 646 of the 3,938 reference mass spectrum data are analyzed by PCR to make sure that the Klebsiella pneumoniae includes the gene encoding the carbapenemases or not and the genotypes thereof. In order to further confirm the genotype type of the Klebsiella pneumoniae, the isolates are cultured overnight in Tryptone Soy Broth at 37° C. for DNA extraction, performed by QlAamp UCP Pathogen Mini Kit (QIAGEN, Hilden, Germany). The carbapenemase-encoding genes, including bla_{IMP}, bla_{VIM}, bla_{spM}, bla_{KPC}, bla_{NDM}, bla_{BIC}, bla_{oxA-48-like}, bla_{AIM}, bla_{DIM}, bla_{GIM}, and bla_{SIM} are analyzed by PCR. Furthermore, the carbapenemase detection by immunochromatographic assay is carried out using NG-Test CARBA 5 (NG Biotech, Guipry, France) to further confirm the carbapenemase types, and the results are shown in Table 2.

TABLE 2

| Genotype of carbapenemase | Sample number |
|---|---|
| KPC | 290 |
| OXA^{a} | 90 |
| VIM | 11 |
| NDM | 13 |
| IMP | 12 |
| Multiple phenotypes | KPC + OXA^{a} = 6 |
| | KPC + VIM = 2 |
| | KPC + NDM = 11 |
| | KPC + IMP = 4 |
| | OXA^{a} + IMP = 4 |
| | OXA^{a} + NDM = 3 |
| | VIM + IMP = 1 |
| | VIM + NDM = 1 |
| ND^{b} | 198 |
| Total | 646 |

OXA^{a} = OXA-48-like, ND^{b} = not detectable, isolates without KPC, OXA-48-like, VIM, NDM or IMP detection.

II. Reference Spectrum Pre-Processing

In the present test, Python (version 3.7) is served as the spectrum pre-processing module of the present disclosure to process the raw signals of the reference mass spectrum data in the database.

First, each of the reference mass spectrum data will be examined initially by the spectrum pre-processing module, and the reference mass spectrum data including blank portions or error formats will be removed. Then, the signals of each of the reference mass spectrum data will be smoothed by the calibration unit of the spectrum pre-processing module so as to remove the background noise thereof.

Next, the raw signal resolution and the sampling frequency of different reference mass spectrum data will be checked by the sampling normalization unit of the spectrum pre-processing module so as to make sure that there is an inconsistency or not. If so, all the reference mass spectrum data will be resampled so as to make the temporal resolution values thereof consistent. Further, the baseline of each of the reference mass spectrum data will be corrected by the top-hat method and then normalized according to the aforementioned Formula (I) so as to normalize the signal intensity. Thus, the signal resolution of all the reference mass spectrum data can be consistent and a plurality of processed reference mass spectrum data can be obtained.

Then, the processed reference mass spectrum data will be processed by a mass-to-charge ratio conversing method by the spectrum conversion module, and a data interval value of each of the processed reference mass spectrum data will be adjusted, wherein the data interval value can be 10 daltons. Therefore, the shift of the peaks of each of the processed reference mass spectrum data or the shifts of the peaks between different processed reference mass spectrum data can be avoided, and a plurality of normalized reference mass spectrum data can be obtained.

After the reference spectrum pre-processing is finished, each of the normalized reference mass spectrum data will be further labeled with the information of Ambler classification, the kinds of carbapenemases and the genotype thereof, and the normalized reference mass spectrum data will be served as the base for analyzing the drug-resistant Klebsiella pneumoniae.

III. Establishing the Antibiotic Resistance Assessing Classifier

The normalized reference mass spectrum data obtained by the reference spectrum pre-processing will be classified according to the Ambler classification thereof, namely KPC (Ambler class A); NDM, VIM and IMP (Ambler class B); and OXA (Ambler class D), as well as the types of the drug-resistant Klebsiella pneumoniae (CSKP and CnSKP as well as CoIRKP and ColIKP). Then, the normalized reference mass spectrum data will be trained to achieve a convergence by the LightGBM algorithm classifier so as to obtain an antibiotic resistance assessing classifier of the present disclosure. Thus, the robustness and the generality of the antibiotic resistance assessing classifier can be maintained, and the antibiotic resistance assessing classifier will be used to analyze the importance of different feature marks of the target mass spectrum data to the assessment of the drug-resistant Klebsiella pneumoniae. Then, the efficiency of the method for assessing drug-resistant Klebsiella pneumoniae and the drug-resistant Klebsiella pneumoniae assessing system of the present disclosure used to assess the drug-resistant Klebsiella pneumoniae based on different anti-carbapenem feature marks or the anti-colistin feature mark of the present disclosure can be illustrated.

IV. The efficacy of the method for assessing drug-resistant Klebsiella pneumoniae and the drug-resistant Klebsiella pneumoniae assessing system of the present disclosure used to assess the antibiotic susceptibility of the drug-resistant Klebsiella pneumoniae to the carbapenem antibiotics

After the target mass spectrum data is analyzed by the drug-resistant Klebsiella pneumoniae assessing system of the present disclosure according to the method for assessing drug-resistant Klebsiella pneumoniae of the present disclosure, the accuracy to distinguish the Klebsiella pneumoniae of the three Ambler classifications, CSKP and CnSKP is 0.8869, the AUROC (area under the receiver operating characteristic curve) is up to 0.9551, and the F1 score can reach 0.8703. Thus, it is shown that the method for assessing drug-resistant Klebsiella pneumoniae and the drug-resistant Klebsiella pneumoniae assessing system of the present disclosure can effectively assess whether the Klebsiella pneumoniae is resistant or susceptible to the carbapenem antibiotics or not.

Further, in the present test, the SHapely Additive exPlanations method (“SHAP method” hereafter) is applied for illustrating the contributions of 10 feature marks having the mass-to-charge ratios with different ranges used to assess the antibiotic susceptibility of the drug-resistant Klebsiella pneumoniae to the carbapenem antibiotics. The mass-to-charge ratios of the 10 feature marks are shown in Table 3, and the sequence in order from the top to the bottom shown in Table 3 presents the importance of the 10 feature marks.

TABLE 3

| Feature marks | Mass-to-charge ratio (Da) |
|---|---|
| 4520 | 4,520-4,529 |
| 6150 | 6,150-6,159 |
| 2180 | 2,180-2,189 |
| 5280 | 5,280-5,289 |
| 7310 | 7,310-7319 |
| 5820 | 5,820-5,829 |
| 3040 | 3,040-3,049 |
| 2750 | 2,750-2,759 |
| 2630 | 2,630-2,639 |
| 6090 | 6,090-6099 |

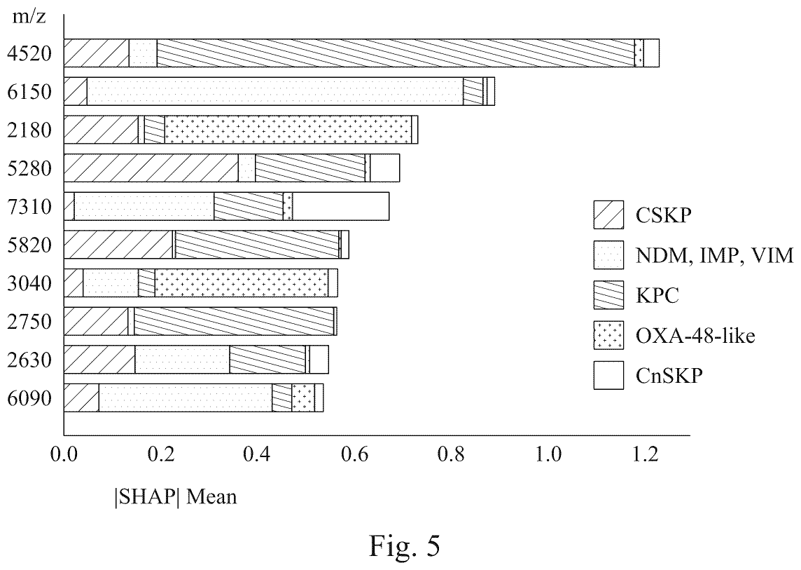

Reference is made to FIG. 5, which shows the analysis results of the contributions of different feature marks to the drug susceptibility analysis program when assessing the susceptibility of the Klebsiella pneumoniae. In FIG. 5, the CnSKP thereof is the isolates without KPC, NDM, VIM, or OXA-48-like carbapenase detections in the NG-Test CARBA 5.

In detail, in the results of SHAP method, each of the feature marks independently presents the contribution thereof to the assessment of the drug susceptibility and does not affect each other's contribution. As shown in FIG. 5, the peak of Feature mark 4520 (the mass-to-charge ratio thereof ranges from 4,520 to 4,529 daltons) has the highest SHAP value, and thus Feature mark 4520 is used as the first anti-carbapenem feature mark of the present disclosure, wherein the first anti-carbapenem feature mark is the most important feature for assessing the drug susceptibility of the Klebsiella pneumoniae. Further, the peak of Feature mark 6150 (the mass-to-charge ratio thereof ranged from 6,150 to 6,159 daltons) has the next highest SNAP value, and the peak of Feature mark 2180 (the mass-to-charge ratio thereof ranges from 2,180 to 2,189 daltons) has the third highest SNAP value. Thus, Feature mark 6150 and Feature mark 2180 are respectively used as the second anti-carbapenem feature mark and the third anti-carbapenem feature mark of the present disclosure.

Further, as shown in FIG. 5, the analyzed results obtained based on SNAP method and the analyzed results of the anti-drug genotypes show that Feature mark 4520 can be used to assess the CnSKP of Ambler class A expressing KPC, Feature mark 6150 can be used to assess the CnSKP of Ambler class B expressing NDM, VIM and IMP, and Feature mark 2180 can be used to assess the CnSKP of Ambler class D expressing OXA-48-like.

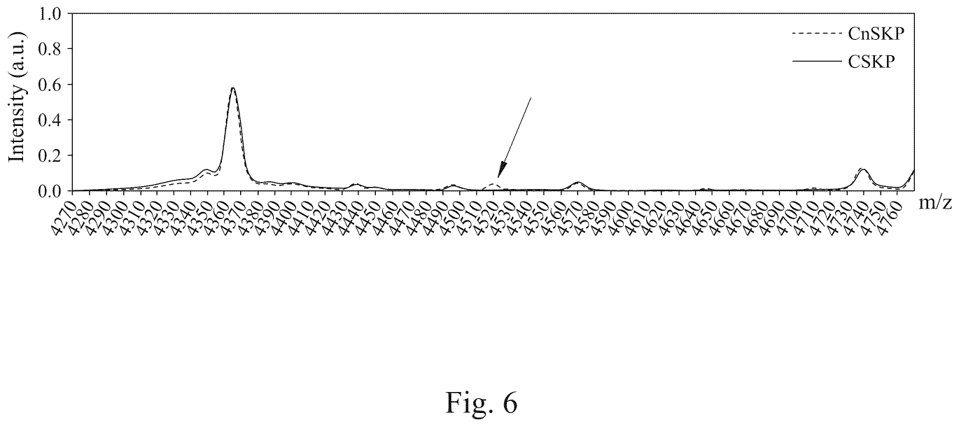

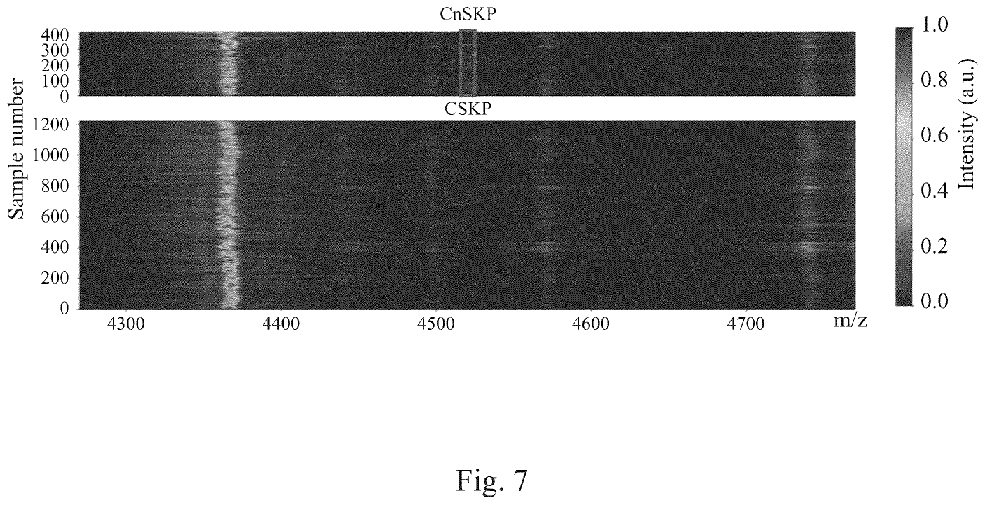

Reference is made to FIG. 6 and FIG. 7. FIG. 6 shows the mass spectrometry data of different types of Klebsiella pneumoniae, and FIG. 7 is a pseudogel plot of all samples used to assess the susceptibility of the Klebsiella pneumoniae to the carbapenem antibiotics. As shown in FIG. 6, the x-axis represents the mass-to-charge ratio value of the mass spectrum data, the y-axis represents the intensity of the peak, and compared with the mass spectrum data of CSKP, the mass spectrum data of CnSKP has a peak with the mass-to-charge ratio of 4,520 to 4,529 daltons. Further, as shown in FIG. 7, the x-axis represents the mass-to-charge ratio value of the mass spectrum data, the y-axis represents the sample number, and compared with the samples of CSKP, the samples of CnSKP have a signal performance in the mass-to-charge ratio of 4,520 to 4,529 daltons (marked in red box).

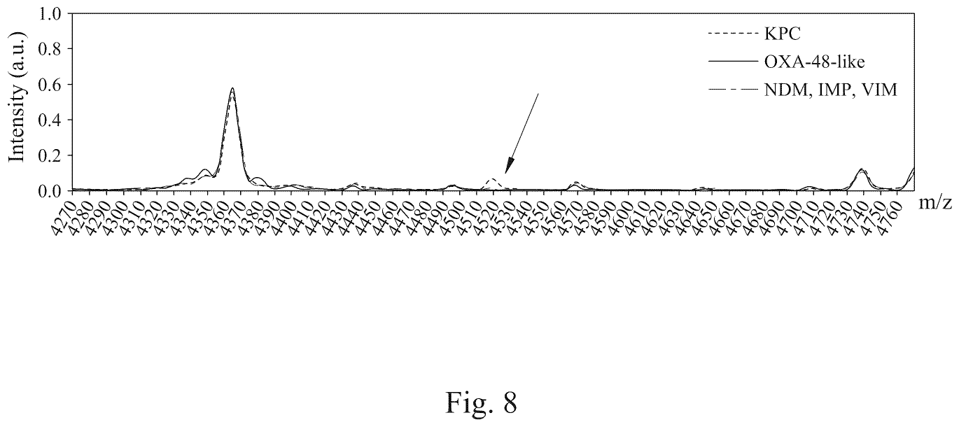

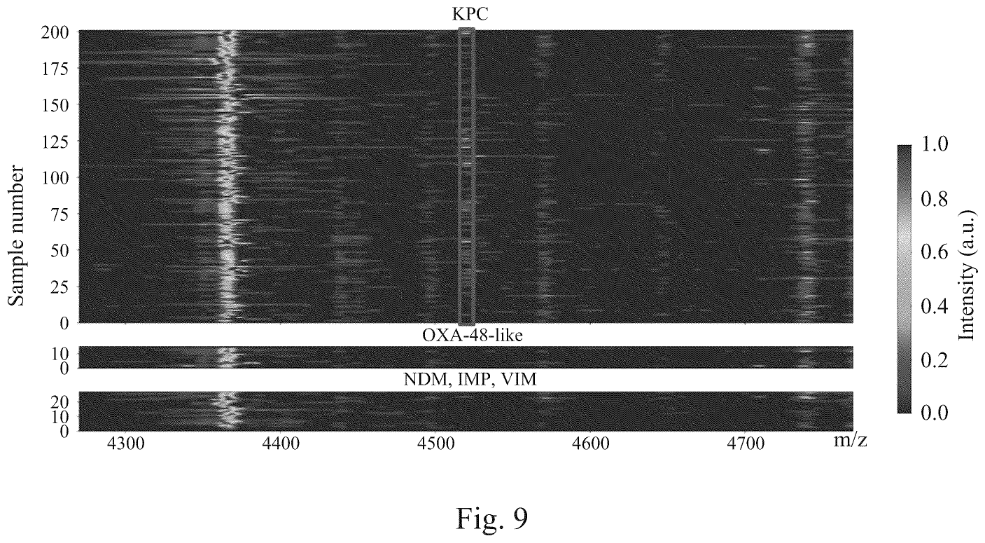

Reference is made to FIG. 8 and FIG. 9. FIG. 8 shows the mass spectrometry data of different types of CnSKP, and FIG. 9 is a pseudogel plot of all samples used to assess the CnSKP of different Ambler classifications. In detail, in the present test, the mass spectrometry data of CnSKP of different Ambler classifications are analyzed simultaneously so as to analyze the performance of the signals thereof. As shown in FIG. 8, compared with the CnSKP of Ambler class B expressing NDM, VIM and IMP as well as CnSKP of Ambler class D expressing OXA-48-like, the mass spectrometry data of CnSKP of Ambler class A expressing KPC has a peak with the mass-to-charge ratio of 4,520 to 4,529 daltons. Further, as shown in FIG. 9, compared to the samples from other types, the samples of CnSKP expressing KPC have a signal performance in the mass spectrometry data thereof with the mass-to-charge ratio of 4,520 to 4,529 daltons (marked in red box).

Therefore, the method for assessing drug-resistant Klebsiella pneumoniae and the drug-resistant Klebsiella pneumoniae assessing system of the present disclosure can efficiently identify the samples including the first anti-carbapenem feature mark with the mass-to-charge ratio of 4,520 to 4,529 daltons based on the raw data of the mass spectrometry data thereof during the assessing process. Accordingly, the present disclosure can be used to assess whether the Klebsiella pneumoniae is resistant to the carbapenem antibiotics or not, the accuracy thereof can be greatly enhanced, and the present disclosure has application potential in related markets.

V. The efficacy of the method for assessing drug-resistant Klebsiella pneumoniae and the drug-resistant Klebsiella pneumoniae assessing system of the present disclosure used to assess the antibiotic susceptibility of the drug-resistant Klebsiella pneumoniae to the colistin

After the target mass spectrum data is analyzed by the drug-resistant Klebsiella pneumoniae assessing system of the present disclosure according the method for assessing drug-resistant Klebsiella pneumoniae of the present disclosure, the accuracy to distinguish CoIRKP and ColIKP is 0.8361, AUROC is up to 0.8447, and the F1 score can reach 0.8925. Thus, it is shown that the method for assessing drug-resistant Klebsiella pneumoniae and the drug-resistant Klebsiella pneumoniae assessing system of the present disclosure can effectively assess whether the Klebsiella pneumoniae is resistant or susceptible to the colistin or not.

Further, in the present test, SHAP method is also applied for illustrating the contributions of 10 feature marks having the mass-to-charge ratios with different ranges used to assess the antibiotic susceptibility of the drug-resistant Klebsiella pneumoniae to the colistin. The mass-to-charge ratios of the 10 feature marks are shown in Table 4, and the sequence in order from the top to the bottom shown in Table 4 presents the importance of the 10 feature marks.

TABLE 4

| Feature mark | Mass-to-charge ratio (Da) |
|---|---|
| 4170 | 4,170-4,179 |
| 7320 | 7,320-7,329 |
| 2240 | 2,240-2,249 |
| 4370 | 4,370-4,379 |
| 2100 | 2,100-2,109 |
| 3130 | 3,130-3,139 |
| 2980 | 2,980-2,989 |
| 3610 | 3,610-3,619 |
| 2840 | 2,840-2,849 |
| 7310 | 7,310-7,319 |

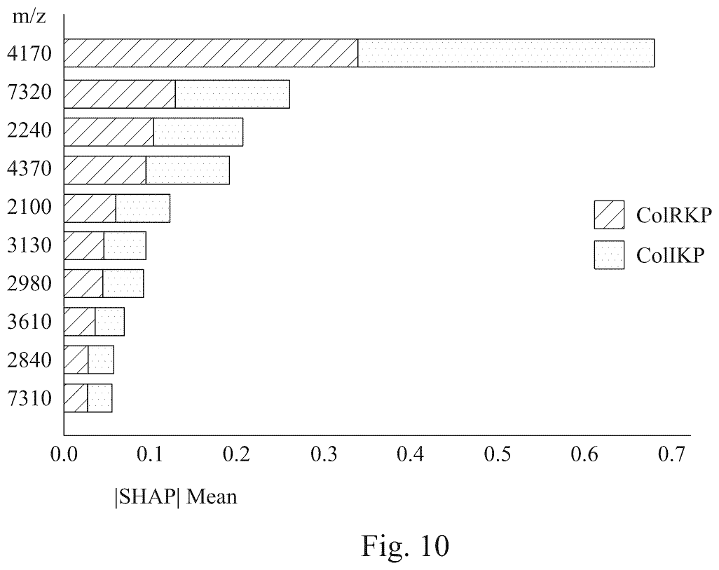

Reference is made to FIG. 10, which shows the analysis results of the contributions of different feature marks to the drug susceptibility analysis program when assessing the susceptibility of the Klebsiella pneumoniae. As shown in FIG. 10, based on the results of SHAP method, the peak of Feature mark 4170 (the mass-to-charge ratio thereof ranges from 4,170 to 4,179 daltons) has the highest SHAP value, the peak of Feature mark 7320 (the mass-to-charge ratio thereof ranges from 7,320 to 7,329 daltons) has the next highest SHAP value, and thus Feature mark 4170 and Feature mark 7320 are respectively used as the first anti-colistin feature mark and the second anti-colistin feature mark of the present disclosure.

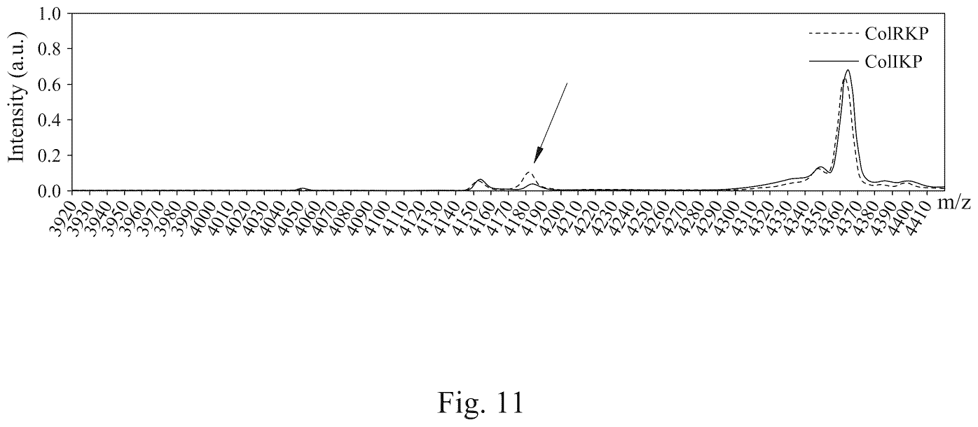

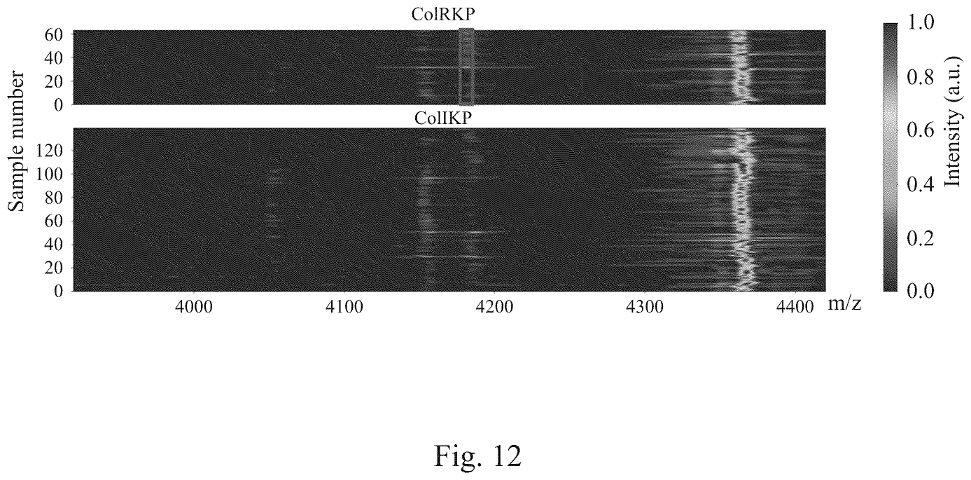

Reference is made to FIG. 11 and FIG. 12. FIG. 11 shows the mass spectrometry data of different types of colistin-resistant Klebsiella pneumoniae, and FIG. 12 is a pseudogel plot of all samples used to assess the susceptibility of Klebsiella pneumoniae to the colistin.

As shown in FIG. 11, compared with the mass spectrum data of ColIKP, the mass spectrometry data of CoIRKP has a peak with the mass-to-charge ratio of 4,170 to 4,179 daltons. Further, as shown in FIG. 12, compared with the samples of CoIRKP, the samples of ColIKP have a signal performance in the mass-to-charge ratio of 4,520 to 4,529 daltons (marked in red box).

Therefore, the method for assessing drug-resistant Klebsiella pneumoniae and the drug-resistant Klebsiella pneumoniae assessing system of the present disclosure can efficiently identify the samples including the first anti-colistin feature mark with the mass-to-charge ratio of 4,170 to 4,179 daltons based on the raw data of the mass spectrometry data thereof during the assessing process. Accordingly, the present disclosure can be used to assess whether the Klebsiella pneumoniae is resistant to the colistin or not, the accuracy thereof can be greatly enhanced, and the present disclosure has application potential in related markets.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A method for assessing drug-resistant Klebsiella pneumoniae, comprising:
providing a test sample, wherein the test sample comprises a Klebsiella pneumoniae;
performing a spectrum analysis step, wherein the test sample is detected by a mass spectrometry method so as to obtain a target mass spectrum data, and the target mass spectrum data is a mass spectrum data of the Klebsiella pneumoniae; and
performing an assessing step for drug-resistant Klebsiella pneumoniae, wherein the target mass spectrum data is analyzed so as to assess whether the Klebsiella pneumoniae is resistant to a carbapenem antibiotic or not;
wherein when the Klebsiella pneumoniae is resistant to the carbapenem antibiotic, the target mass spectrum data comprises a first anti-carbapenem feature mark, and a mass-to-charge ratio of the first anti-carbapenem feature mark ranges from 4,520 to 4,529 daltons.

2. The method of claim 1, wherein when the target mass spectrum data comprises the first anti-carbapenem feature mark, the Klebsiella pneumoniae is a carbapenem resistant Klebsiella pneumoniae and comprises a Klebsiella pneumoniae carbapenemase (KPC).

3. The method of claim 1, wherein when the Klebsiella pneumoniae is resistant to the carbapenem antibiotic, the target mass spectrum data further comprises:
a second anti-carbapenem feature mark, wherein a mass-to-charge ratio of the second anti-carbapenem feature mark ranges from 6,150 to 6,159 daltons.

4. The method of claim 3, wherein when the target mass spectrum data comprises the second anti-carbapenem feature mark, the Klebsiella pneumoniae comprises a metallo-β-lactamases (MBL), and the metallo-β-lactamase is imipenemase MBL (IMP), New Delhi MBL (NDM), or Verona integron-encoded MBL (VIM).

5. The method of claim 1, wherein when the Klebsiella pneumoniae is resistant to the carbapenem antibiotic, the target mass spectrum data further comprises:
a third anti-carbapenem feature mark, wherein a mass-to-charge ratio of the third anti-carbapenem feature mark ranges from 2,180 to 2,189 daltons.

6. The method of claim 5, wherein when the target mass spectrum data comprises the third anti-carbapenem feature mark, the Klebsiella pneumoniae comprises an oxacillinase-type carbapenemase (OXA), and the oxacillinase-type carbapenemase is OXA-48-like carbapenemases.

7. A method for assessing drug-resistant Klebsiella pneumoniae, comprising:
providing a test sample, wherein the test sample comprises a Klebsiella pneumoniae;
performing a spectrum analysis step, wherein the test sample is detected by a mass spectrometry method so as to obtain a target mass spectrum data, and the target mass spectrum data is a mass spectrum data of the Klebsiella pneumoniae; and
performing an assessing step for drug-resistant Klebsiella pneumoniae, wherein the target mass spectrum data is analyzed so as to assess whether the Klebsiella pneumoniae is resistant to a colistin or not;
wherein when the Klebsiella pneumoniae is resistant to the colistin, the target mass spectrum data comprises a first anti-colistin feature mark, and a mass-to-charge ratio of the first anti-colistin feature mark ranges from 4,170 to 4,179 daltons.

8. The method of claim 7, wherein when the Klebsiella pneumoniae is resistant to the colistin, the target mass spectrum data further comprises:
a second anti-colistin feature mark, wherein a mass-to-charge ratio of the second anti-colistin feature mark ranges from 7,320 to 7,329 daltons.

9. A drug-resistant Klebsiella pneumoniae assessing system, comprising:
a non-transitory machine readable medium for storing a target mass spectrum data, wherein the target mass spectrum data is obtained by detecting a test sample with a mass spectrometry method, and the test sample comprises a Klebsiella pneumoniae; and
a processor signally connected to the non-transitory machine readable medium, wherein the processor comprises:
an analyzing module for analyzing the target mass spectrum data by a drug susceptibility analysis program so as to output a feature set, wherein the drug susceptibility analysis program is for assessing whether the Klebsiella pneumoniae is resistant to a carbapenem antibiotic or a colistin or not based on the feature set;
wherein the feature set comprises a first anti-carbapenem feature mark and a first anti-colistin feature mark, a mass-to-charge ratio of the first anti-carbapenem feature mark ranges from 4,520 to 4,529 daltons, and a mass-to-charge ratio of the first anti-colistin feature mark ranges from 4,170 to 4,179 daltons;
wherein when the Klebsiella pneumoniae is resistant to the carbapenem antibiotic, the target mass spectrum data comprises the first anti-carbapenem feature mark;
wherein when the Klebsiella pneumoniae is resistant to the colistin, the target mass spectrum data comprises the first anti-colistin feature mark.

10. The drug-resistant Klebsiella pneumoniae assessing system of claim 9, wherein when the target mass spectrum data comprises the first anti-carbapenem feature mark, the Klebsiella pneumoniae comprises a Klebsiella pneumoniae carbapenemase.

11. The drug-resistant Klebsiella pneumoniae assessing system of claim 9, wherein the feature set further comprises a second anti-carbapenem feature mark, and when the Klebsiella pneumoniae is resistant to the carbapenem antibiotic, the target mass spectrum data further comprises the second anti-carbapenem feature mark, and a mass-to-charge ratio of the second anti-carbapenem feature mark ranges from 6,150 to 6,159 daltons.

12. The drug-resistant Klebsiella pneumoniae assessing system of claim 11, wherein when the target mass spectrum data comprises the second anti-carbapenem feature mark, the Klebsiella pneumoniae comprises a metallo-β-lactamases, and the metallo-β-lactamase is imipenemase MBL, New Delhi MBL, or Verona integron-encoded MBL.

13. The drug-resistant Klebsiella pneumoniae assessing system of claim 9, wherein the feature set further comprises a third anti-carbapenem feature mark, and when the Klebsiella pneumoniae is resistant to the carbapenem antibiotic, the target mass spectrum data further comprises the third anti-carbapenem feature mark, and a mass-to-charge ratio of the third anti-carbapenem feature mark ranges from 2,180 to 2,189 daltons.

14. The drug-resistant Klebsiella pneumoniae assessing system of claim 13, wherein when the target mass spectrum data comprises the third anti-carbapenem feature mark, the Klebsiella pneumoniae comprises an oxacillinase-type carbapenemase, and the oxacillinase-type carbapenemase is OXA-48-like carbapenemases.

15. The drug-resistant Klebsiella pneumoniae assessing system of claim 9, wherein the feature set further comprises a second anti-colistin feature mark, and when the Klebsiella pneumoniae is resistant to the colistin, the target mass spectrum data further comprises the second anti-colistin feature mark, and a mass-to-charge ratio of the second anti-colistin feature mark ranges from 7,320 to 7,329 daltons.
